(19) United States
(12) Patent Application Publication
(10) Pub. No.: US 2002/0037495 A1
(43) Pub. Date: Mar. 28, 2002

(54) High throughput screening for inhibitors of fatty acid, ergosterol, sphingolipid, or phospholipid synthesis in fungi

(76) Inventors: Juan Antonio Gutierrez, Natick, MA (US); Christine Ellen Bulawa, Arlington, MA (US); Ronald K. Blackman, Brookline, MA (US); Victoria Gavrias, North Stonington, CT (US)

Correspondence Address: J. PETER FASSE, FISH & RICHARDSON P.C., 225 Franklin Street, Boston, MA, 02110-2804, US

(21) Appl. No.: 09/815,588

(22) Filed: Mar. 23, 2001

Related U.S. Application Data
(60) Provisional application No. 60/191,466, filed on Mar. 23, 2000.

(51) Int. Cl. A61K 31/00; C12Q 1/00; C12Q 1/68; G01N 33/569

(52) U.S. Cl. 435/4; 435/6; 435/7.3; 514/1

(57) ABSTRACT

Methods for identifying compounds that are inhibitors of fatty acid, ergosterol, sphingolipid, or phospholipid synthesis are disclosed. Such compounds can be derivatized to produce antifungal agents, which can be used in methods of treating fungal infections (e.g., in humans, animals, and plants). The disclosed methods allow for high throughput screening of libraries of test compounds.

### CROSS-REFERENCE TO RELATED APPLICATION

This application claims priority from U.S. Provisional Application No. 60/191,466, filed on Mar. 23, 2000, which is incorporated herein by reference in its entirety.

### FIELD OF THE INVENTION

The invention relates to methods for identifying inhibitors of fatty acid synthesis in fungi.

### BACKGROUND OF THE INVENTION

Fatty acid synthesis is necessary for the production of fungal cell membranes, and therefore is essential for survival of fungi. This synthetic pathway has two main events: (i) fatty acid chain extension by the addition of acetyl CoA, and (ii) desaturation by Δ-9 fatty acid desaturase encoded by the gene OLE1. Synthesis of saturated fatty acids utilizes malonyl CoA, which is produced by acetyl CoA carboxylase (Acclp). Malonyl CoA can be used to synthesize ergosterol and fatty acids, both essential components of fungal membranes. The yeast fatty acid synthetase complex is composed of the Fas1p and Fas2p subunits, and the FAS2 gene encodes the trifunctional subunit of fatty acid synthetase of yeast. Fatty acids are further utilized in sphingolipid and phospholipid biosynthesis, both of which are essential for fungal viability.

### SUMMARY OF THE INVENTION

The invention is based upon the discovery that the activity of promoters of certain genes is increased in the presence of a compound that inhibits fatty acid synthesis. Thus, compounds that inhibit fungal fatty acid synthesis can be identified by their ability to increase the activity of the promoters of genes such as OLE1, YOL101c, and YGL039w, which are described herein. Fungal fatty acid synthesis inhibitors that slow the growth of, or kill, fungi are candidate antifungal compounds that may be, or that may be modified or derivatized to be, antifungal agents, which can be used in methods of treating fungal infections. The new methods and compositions thus provide a rapid and convenient method for identifying (i) compounds that inhibit fungal fatty acid synthesis and which can subsequently be modified or derivatized to produce antifungal agents, (ii) compounds that inhibit fungal fatty acid synthesis and which are themselves antifungal agents, and (iii) novel targets.

Accordingly, the invention features a method for determining whether a test compound is an inhibitor of fungal fatty acid, ergosterol, sphingolipid, and/or phospholipid biosynthesis. The method includes contacting a yeast cell with a test compound, wherein the yeast cell contains a promoter (e.g., a promoter from Saccharomyces, or from S. cerevisiae, or the specific OLE1, YOL101c, or YGL039w promoters described herein, or homologs or orthologs of these specific promoters). The main point is that the activity of these promoters must increase in the presence of a compound that inhibits fatty acid synthesis. The promoter is operably linked to a reporter gene, and the method further includes measuring activity of the promoter, relative to the level of activity of the promoter in the absence of the test compound, wherein an increase in promoter activity indicates that the test compound is an inhibitor of fungal fatty acid, ergosterol, sphingolipid, and/or phospholipid synthesis.

A promoter is “operably linked” to a reporter gene if the promoter and gene are connected in such a way as to permit gene expression when appropriate molecules (e.g., transcriptional activator proteins) contact the promoter. “Homologs” are genes that are different, but structurally and functionally similar or equivalent, and are found in the same species. On the other hand, “orthologs” are genes that are different, but structurally and functionally similar or equivalent, and are each found in a different species (within or outside of a given genus).

The invention also includes a method for determining whether a test compound is an antifungal agent. The method includes: (i) contacting a yeast cell with a test compound, wherein the yeast cell contains (a) a promoter, the activity of which is increased in the presence of a compound that inhibits fatty acid synthesis (e.g., the OLE1, YOL101c, or YGL039w promoter), operably linked to (b) a reporter gene; (ii) measuring activity of the promoter, relative to the level of activity of the promoter in the absence of the test compound, wherein an increase in activity indicates that the test compound is a candidate antifungal compound that inhibits fungal fatty acid, ergosterol, sphingolipid, or phospholipid synthesis; and (iii) determining whether the candidate compound inhibits growth of a fungus, wherein an inhibition of fungal growth indicates that the candidate compound is an antifungal agent.

If desired, the candidate compound may be further assayed to determine whether it inhibits fatty acid synthesis. Inhibition of fatty acid synthesis can be detected, for example, as inhibition of incorporation of acetate into fatty acid. Inhibition can be measured in an extract of the cell if desired by measuring the activity of an individual enzyme(s) along the pathway.

Various promoters can be used in the invention provided that the activity of the promoter is increased by an antifungal agent (e.g., a fatty acid synthesis inhibitor such as cerulenin). For example, the promoter can be the promoter of a yeast gene, e.g., a S. cerevisiae gene, such as OLE1, YOL101c, or YGL039w.

An increase in activity of the promoter can be measured, for example, by measuring expression of a reporter gene such as lacZ, cat, gus, luciferase genes, or genes encoding fluorescent markers such as green fluorescent protein. Other suitable reporter genes are well known in the art and can be used in the invention. If desired, the activity of the promoter can be quantified by measuring binding of antibodies to a product of the reporter gene (e.g., a protein encoded by the reporter gene), with an increase in the level of bound antibodies reflecting an increase in the activity of the promoter. Alternatively, activity can be quantified by measuring mRNA transcribed from the reporter gene, with an increase in the MRNA level reflecting an increase in promoter activity.

The invention also provides methods of preparing (i) an inhibitor of fungal fatty acid, ergosterol, sphingolipid, or phospholipid synthesis or (ii) an antifungal agent. The methods include: screening multiple test compounds by the methods described above; identifying candidate compounds that upregulate promoter activity; isolating one or more lead compounds from the candidate compounds; identifying a lead compound that inhibits fatty acid, ergosterol, sphingolipid, or phospholipid synthesis or fungal growth; selecting a lead compound that inhibits fatty acid synthesis or fungal growth; and formulating the selected lead compound as an inhibitor of fatty acid synthesis or as an antifungal agent. A “lead compound” is a test compound that increases promoter activity by at least a statistically significant amount. If desired, lead compounds can be subsequently derivatized using conventional medicinal chemistry methods, as described herein.

Similarly, the invention features methods for preparing (i) an inhibitor of fungal fatty acid synthesis or (ii) an antifungal agent. The methods include screening multiple test compounds by the methods described above; identifying candidate compounds that upregulate promoter activity; isolating one or more lead compounds from the candidate compounds; derivatizing the lead compound(s), thereby producing a derivative of the lead compound; identifying derivatives that inhibit fatty acid synthesis or fungal growth; and formulating the derivative as an inhibitor of fungal fatty acid synthesis or as an antifungal agent (e.g., by admixture with a pharmaceutically acceptable carrier). Inhibitors of fatty acid synthesis and antiflingal agents prepared by such methods also are included within the invention. Such compounds can be used in methods for inhibiting fungal fatty acid synthesis or growth of fungi in an organism having a fungal infection.

The invention offers several advantages. For example, the methods described herein can readily be used in high-throughput screening (HTS) of a wide variety of test compounds. Because of interconnected biochemical pathways and the potential redundancy of induced transcripts upon inhibition of essential genes, the methods disclosed herein enable the identification of compounds that inhibit the products of essential of genes other than Fas2p and Acc1p. Assays employing the OLE1 and YOL101c promoters are capable of detecting fatty acid inhibitors (such as cerulenin) at concentrations both above and below their minimal inhibitor concentration (MIC). In addition, the OLE1 reporter can be used to detect inhibitors of ergosterol, sphingolipid, and phospholipid biosynthesis, since inhibitors of these pathways (such as miconoazole and myriocin) also induce this reporter. Thus, the assays described herein provide a high level of sensitivity and are expected to detect growth inhibitory (i.e., antifungal) compounds, as well as less potent inhibitors of fatty acid, ergosterol, sphingolipid, and phospholipid synthesis, which can subsequently be modified using standard medicinal chemistry techniques and by evaluating structure-activity relationship (SAR) data. Because the assays are cell-based, the assays identify inhibitors that can efficiently enter fungal cells. Therefore, the assays allow the identification of potent antifungal compounds and compounds of structural interest that may have relatively modest potency, but have favorable cell permeability properties.

Unless otherwise defined, all technical and scientific terms used herein have the same meaning as commonly understood by one of ordinary skill in the art to which this invention belongs. Various methods and materials similar or equivalent to those described herein can be used in the practice or testing of the present invention, and examples of suitable methods and materials are described below. All publications, patent applications, patents, technical manuals, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following description of the preferred embodiments thereof, and from the claims.

### BRIEF DESCRIPTION OF THE DRAWINGS

FIG. 1 is a listing of the nucleotide sequence of the OLE1 promote (SEQ ID NO:1).

FIG. 2 is a listing of the nucleotide sequence of the YOL101c promoter (SEQ ID NO:2).

FIG. 3 is a listing of the nucleotide sequence of the YGLO39w promoter (SEQ ID NO:3).

FIG. 4 is a graph of growth curves of S. cerevisiae cells with and without cerulenin induction.

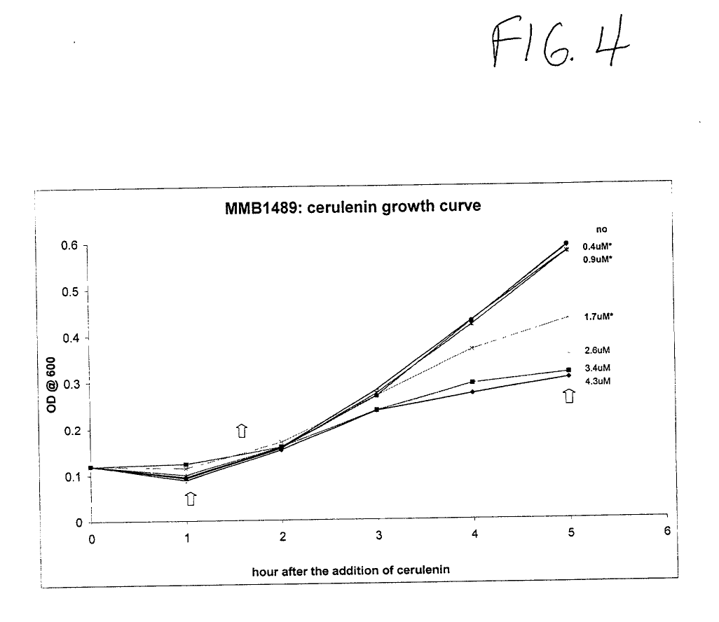

### DETAILED DESCRIPTION

The invention provides methods for determining whether a test compound is an inhibitor of fungal fatty acid synthesis. The invention derives from the discovery that compounds that inhibit fatty acid synthesis cause an increase in the activity of the promoters of genes such as OLE1, YOL101c, and YGL039w. Generally, the method involves determining whether the test compound increases the activity of an OLE1, YOL101c, or YGL039w promoter (i.e., a reporter-based screen). As discussed in detail below, the activity of the OLE1, YOL101c, and YGL039w promoters is increased upon inhibition of the FAS2 gene and its product. Thus, a compound that increases the activity of the OLE1 and/or YOL101c and/or YGL039w promoters can be expected to inhibit fatty acid synthesis, and is thus a candidate antifungal compound. Optionally, the candidate compound can be further tested to confirm that it inhibits fatty acid synthesis (i.e., a biochemical screen), providing a further indication that the compound is an inhibitor of fungal fatty acid, ergosterol, sphingolipid and phospholipid synthesis. Such compounds can subsequently be derivatized, using medicinal chemistry, to produce antifungal agents. These methods are described in further detail below.

Part I: Reporter-Based Screen

Promoters: Using RNA profiling in yeast (described in detail in the Examples section below), the activities of the OLE1, YOL101c, and YGL039w promoters in yeast were shown to be increased by the antifungal agent cerulenin, which is an inhibitor of Fas2p. In an alternative method, increased promoter activity was also detected by using a genetic, rather than chemical, method to inhibit fatty acid synthesis. In this method, a “shut off” strain was used to decrease, in a regulatable manner, the expression of a gene required for fatty acid synthesis (e.g., FAS2 or ACC1). The strain KTYFAS4 (Millennium Pharmaceuticals Inc.; MMB #1549) served as a shut off strain for the FAS2 gene, and the strain KTYAC (Millennium Pharmaceuticals Inc.; MMB#1551) served as a shut off strain for ACC1. The activity of the OLE1, YOL101c, and YGL039w promoters was increased in these shut off strains when expression of the FAS2 or ACC1 genes was shut off, thereby indicating that the activity of the promoters would be increased in the presence of a compound that inhibits fatty acid synthesis.

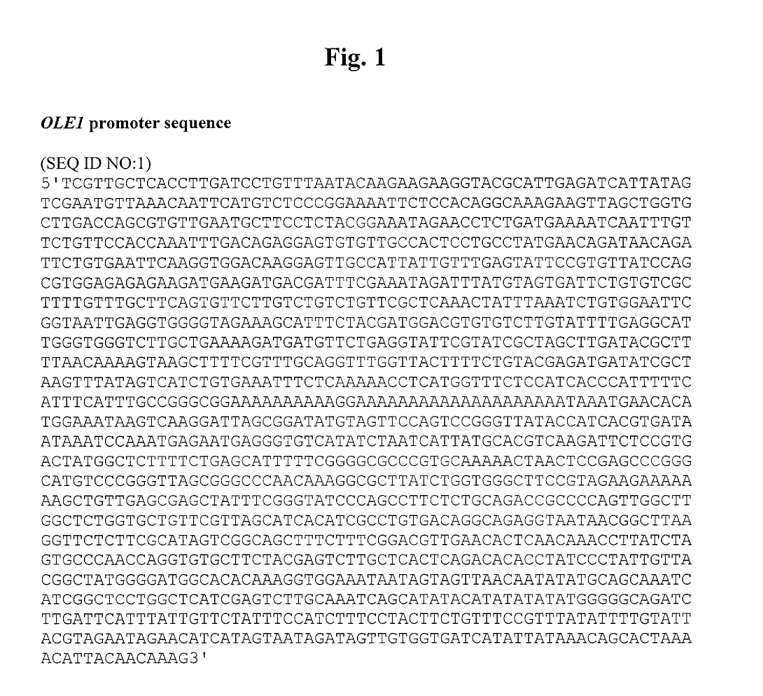

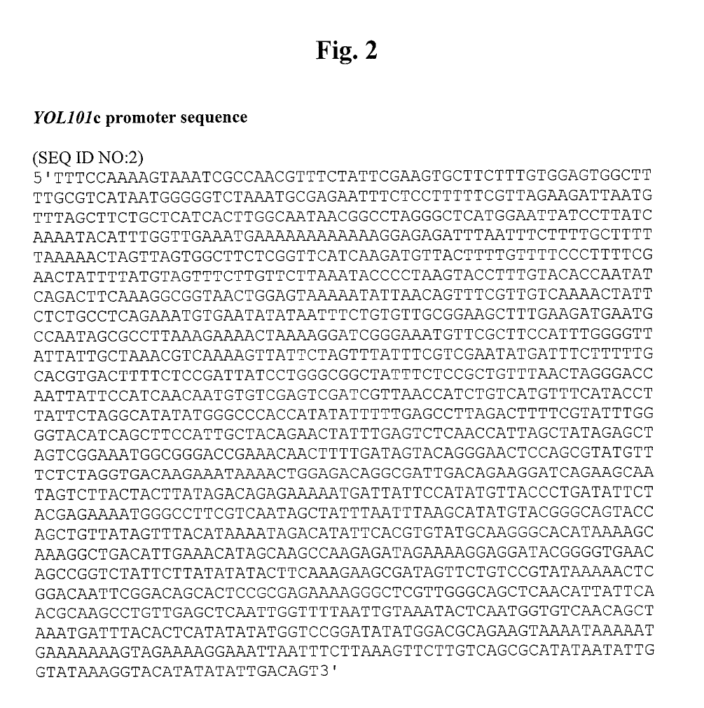

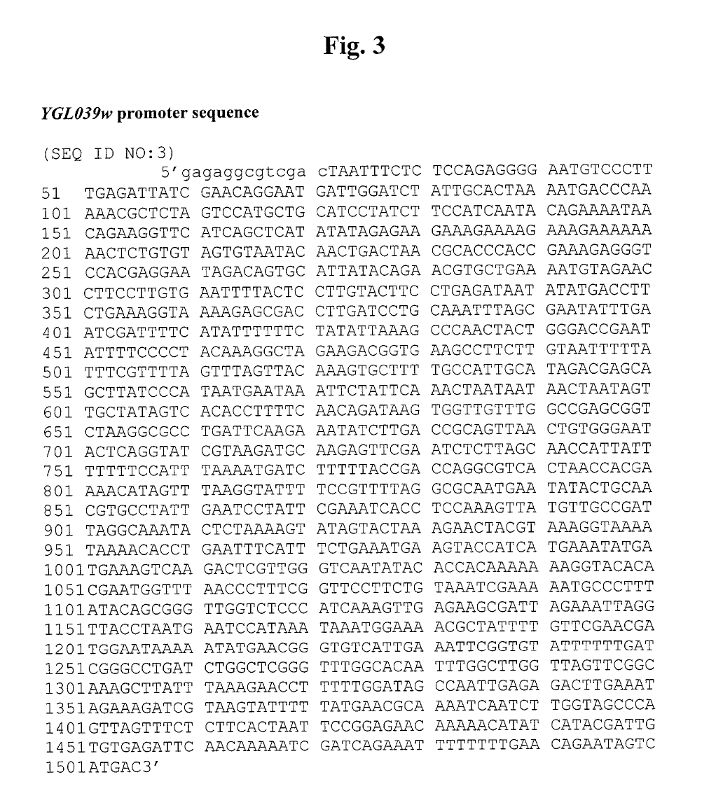

A “promoter” is a minimal sequence sufficient to direct transcription; the promoter can be located in the 5' region of the native gene. A sequence containing the OLE1 promoter is set forth in FIG. 1 as SEQ ID NO: 1. A sequence containing the YOL101c promoter is set forth in FIG. 2 as SEQ ID NO:2. A sequence containing the YGLO39w promoter is set forth in FIG. 3 as SEQ ID NO:3. The ability of a particular sequence to direct gene expression, or to be upregulated by an antifungal agent such as cerulenin, can be tested using methods such as those described herein or methods well known in the art.

Reporter Genes: A reporter gene can be operably linked to the OLE1, YOL101c, or YGL039w promoter to provide a convenient means for measuring an increase in the activity of the promoter. The “reporter gene” can be any sequence for which expression can be detected or measured, other than the coding sequence to which the promoter naturally is operably linked. Examples of suitable reporter genes include, without limitation, lacZ, the bacterial chloramphenicol transacetylase (cat) gene, luciferase genes, and the bacterial gus gene. Also included are genes that encode fluorescent markers, such as the green fluorescent protein (GFP) gene. The aforementioned genes, and methods for measuring their expression, are well known in the art.

Genetic Constructs: The OLE1, YOL101c, or YGL039w promoter and the reporter gene can be included on a genetic construct that can be introduced into a cell. A variety of genetic constructs appropriate for gene expression are well known in the art and can be used in the invention. For example, the promoter and reporter gene can be included on a vector such as pRS415 (Sikorski et al., Genetics 122:19-27), e.g., by cloning the promoter and reporter sequences into the SalI/BamHI sites of the vector. Other vectors that function in yeast also can be used in the invention. If desired, a reporter gene lacking an ATG translation start site can be cloned into a vector, and an ATG translation start site then can be provided by including the ATG translation start site of the gene from which the promoter is obtained. Such vectors are appropriate for expression in yeast, and can readily be introduced into yeast cells according to conventional methods. Examples of suitable yeast strains include PL1 and P41, which are the KTYPS131 strain (Millennium Pharmaceuticals Inc., Myco Pharmaceuticals; MMB#1489; relevant genotype=MAT a/MAT α his3Δ1/his3Δ1 leu2Δ0/leu2Δ0 LYS2/lys2Δ0 met15 Δ0/MET15Δ0 ura3Δ0/ura3Δ0 pdr5Δ::HIS3/pdr5Δ::HIS3 snq2Δ::HIS3/snq2Δ::HIS3) transformed with the pRS415 OLE1p::lacZ and pRS415 YOL101cp::lacZ constructs, respectively. If desired, the promoter and reporter gene can be integrated into the chromosome of a yeast cell.

Test Compounds: Essentially any compound can be used as the test compound. The test compound can be, without limitation, a small organic or inorganic molecule, amino acid, polypeptide, protein, nucleic acid, peptide nucleic acid, carbohydrate, or polysaccharide. The test compound can be synthetic, naturally occurring, or a combination of synthetic and natural components. If desired, the test compound can be a member of a library of test compounds (e.g., a combinatorial chemical library or “diversity” library or “file”) or a component of a cellular extract or bodily fluid (e.g., urine, blood, tears, sweat, or saliva).

Assay of Promoter Induction: Test compounds that increase the activity of the OLE1, YOL101c, or YGL039w promoters, relative to the level of promoter activity in the absence of the test compound, are considered inhibitors of fungal fatty acid synthesis. An increase in promoter activity can be detected by culturing, in the presence of the test compound, a cell containing the OLE1, YOL101c, or YGL039w promoter operably linked to a reporter gene. The level of promoter activity, as evidenced by the level of reporter gene expression obtained in the presence of the test compound, then is compared with the level obtained in the absence of the test compound.

Promoter activity, as measured by reporter gene expression, can be measured by any of a number of conventional methods, and the optimal method will depend upon factors such as the nature and function of the reporter gene. In general, methods that permit high throughput screening of numerous test compounds are preferred. Examples of suitable assays of reporter gene expression include methods such as (i) assaying the function of a product of the reporter gene (e.g., measuring an enzymatic reaction catalyzed by a product of the reporter gene); (ii) measuring the level of protein expressed from the reporter gene (e.g., by SDS-PAGE or in an immunoassay using antibodies (e.g., polyclonal or monoclonal antibodies) that specifically bind to the product of the reporter gene); and (iii) measuring the level of mRNA transcribed from the reporter gene.

The assays of promoter induction can be carried out in virtually any reaction vessel or receptacle. Examples of suitable receptacles include 96-well plates, 384-well plates, test tubes, centrifuge tubes, and microcentrifuge tubes. The methods can also be carried out on surfaces such as metal, glass, ceramics, paper, polymeric chips, membrane surfaces, resins, or the surface of a matrix-assisted laser-desorption ionization mass spectrometry (MALDI-MS) plate.

Generally, an increase in the level of promoter activity that is statistically significant indicates that the test compound is an inhibitor of fungal fatty acid synthesis. An increase that is at least two-fold (e.g., 5-fold, 10-fold, 20-fold, 50-fold, or even 100-fold or greater), relative to the level of gene expression in the absence of the test compound, indicates that the test compound inhibits fungal fatty acid, ergosterol, sphingolipid, or phospholipid synthesis, and is thus a candidate antifungal compound, i.e., a compound (or derivative thereof) that may be an antifungal agent that is able to kill, or slow the growth of, fungi. A relatively high level of induction indicates that the test compound has a relatively high level of potency.

Part II: Biochemical Screen

Once a test compound is identified as an inhibitor of fatty acid, ergosterol, sphingolipid, or phospholipid synthesis using the above-described reporter-based assay, the candidate compound can be further tested, if desired, in a biochemical assay. Such a biochemical assay generally involves measuring inhibition of fatty acid, ergosterol, sphingolipid or phospholipid synthesis. Various methods for measuring inhibition of fatty acid synthesis are known in the art, and can be used in this aspect of the invention (see, e.g., Buttke et al., Biochemistry, 17:5282-5286, 1978; Kuhajda et al., P.N.A.S., 91:6379-6383, 1994; Weiss et al., Biol. Chem. Hoppe Seyler, 367:905-912, 1986; Stoops et al., J. Biol. Chem., 253:4464-4475, 1978; Duronio et al., J. Cell Biol., 113:1313-1330, 1991; Meyer et al., 117:345-350, 1974; Zhao et al., Microbiol., 142:2509-2514, 1996; and Schneiter et al., Mol. Cell. Biol., 16:7161-7172, 1996, which are incorporated herein by reference). For example, the compound can be tested for its ability to inhibit incorporation of ¹⁴C-acetate into fatty acid or ergosterol. Compounds that inhibit incorporation of acetate into fatty acid can be used as inhibitors of fungal fatty acid or ergosterol synthesis. There are other methods to monitor inhibition of sphingolipid and phosholipid in labeling experiments using ortho-[³³P]-phosphate (Steiner et al., Biochim. et Biophys. Acta, 260:82-87).

If desired, inhibition of fatty acid synthesis can be measured in a cell extract that contains all of the components (e.g., enzymes, cofactors, carrier molecules, and buffers) normally necessary for a particular step that is needed in the synthesis of fatty acids. Cell extracts containing enzymes, cofactors, and carrier molecules can be cytoplasmic, cytosolic, or membrane preparations, whole cells, or naturally-occurring or synthetic mixtures composed of natural or unnatural components, or both. Carrier molecules included in the cell extract can include numerous components, such as molecular transport machinery and membranes. The substrate for the reaction can be contained within the cell extract initially, it can be added in solution as a liquid or dry additive or suspension or dispersion, or it can be generated in situ (e.g., as the product of another reaction). The substrate can be detectably labeled with a tag, for example, a radiolabel, a fluorescent label, a magnetic label, or as a biotinylated derivative. After incubation of the cell extract/substrate mixture under conditions that normally allow the particular step(s) to proceed, the mixture is assayed to determine whether the substrate remains and/or whether the corresponding product or products have been formed. The optimal duration of incubation varies with the particular synthesis step(s) being carried out and also with incubation temperature (e.g., 1 hour, 12 hours, 1 day, 2 days, a week, or longer, at, e.g., room temperature or lower, 30° C., 37° C., or higher, depending on the strain of yeast).

Medicinal Chemistry

Once a compound has been identified as an inhibitor of fatty acid (or other) synthesis, principles of standard medicinal chemistry can be used to produce derivatives of the candidate compound. The moieties that are responsible for the compound's activity can be revealed by examining its structure-activity relationships (SAR). Specifically, a person of ordinary skill in the art of chemistry could modify a moiety of the compound to study the effects of the modification on the potency of the compound and thereby produce derivatives of the compound having increased potency (See, e.g., Nagarajan et al., Antibiot., 41:430-438). For example, chemical modifications such as N- acylation, esterification, hydroxylation, alkylation, amination, amidation, oxidation, or reduction can be made. Such chemical modifications can be made according to conventional methods (See, e.g., Wade, Organic Chemistry, Prentice-Hall, Inc., New Jersey, 1987). If structural information regarding the fatty acid synthesis inhibitor and its target are available, derivatives of the inhibitor can be generated and optimized virtually by using molecular modeling software and conventional methods. Such software is commercially available (e.g., from Tripos Inc., Molecular Simulations, Inc., and MDL Information Systems, Inc).

Use of Inhibitors of Fungal Fatty Acid, Ergosterol, Sphingolipid, and Phospholipid Synthesis

A compound identified as an inhibitor of fungal fatty acid, ergosterol, sphingolipid, or phospholipid synthesis, e.g., a candidate compound, or as an antifungal agent can be used to treat a fungal infection in an organism (e.g., an animal, e.g., a mammal, such as a human, dog, cat, cow, horse, or pig, or poultry such as chickens, or a plant). Because genes involved in the synthesis of fatty acid, ergosterol, sphingolipid, or phospholipid are widely conserved, inhibitors of fatty acid biosynthesis in yeast or other specific fungi are expected to be useful in inhibiting fatty acid biosynthesis in a wide spectrum of fungi. For example, the inhibitors are expected to be useful in inhibiting fatty acid synthesis in pathogenic fungi, such as Candida ssp. (e.g., Candida albicans), Cryptococcus ssp. (e.g., Cryptococcus neoformans), and Aspergillus spp. (e.g., Aspergillus fumigatus), and/or non-pathogenic fungi, such as Saccharomyces ssp. (e.g., Saccharomyces cerevisiae).

A therapeutically effective amount of the inhibitor or antifungal agent, formulated as a pharmaceutical composition, can be administered to the organism in a method of treatment (e.g., topically, orally, nasally, buccally, subcutaneously, or intraperitoneally). An “effective amount” of an inhibitor or antifungal agent is an amount sufficient to alleviate a sign(s) and/or symptom(s) of a fungal infection. Treatment typically includes administering a pharmaceutically effective amount of a composition containing an inhibitor(s) or antifungal agent(s) to a subject in need of such treatment, thereby inhibiting fungal growth in the subject. Such a composition typically contains from about 0.1 to 90% by weight (such as 1 to 20% or 1 to 10%) of the active ingredient(s) in a pharmaceutically acceptable carrier.

Solid formulations of the compositions for oral administration may contain suitable carriers or excipients, such as corn starch, gelatin, lactose, acacia, sucrose, kaolin, mannitol, dicalcium phosphate, calcium carbonate, sodium chloride, or alginic acid. Disintegrators that can be used include, without limitation, micro-crystalline cellulose, corn starch, sodium starch glycolate, and alginic acid. Tablet binders that may be used include acacia, methylcellulose, sodium carboxymethylcellulose, polyvinylpyrrolidone (Povidone™), hydroxypropyl methylcellulose, sucrose, starch, and ethylcellulose. Lubricants that may be used include magnesium stearates, stearic acid, silicone fluid, talc, waxes, oils, and colloidal silica.

Liquid formulations of the compositions for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation into the lungs of a mammal to be treated.

Injectable formulations of the compositions may contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water soluble versions of the compounds may be administered by the drip method, whereby a pharmaceutical formulation containing the compound and a physiologically acceptable excipient is infused. Physiologically acceptable excipients may include, for example, 5% dextrose, 0.9% saline, Ringer's solution or other suitable excipients. For intramuscular preparations, a sterile formulation of a suitable soluble salt form of the compounds, can be dissolved and administered in a pharmaceutical excipient such as Water-for-Injection, 0.9% saline, or 5% glucose solution. A suitable insoluble form of the compound may be prepared and administered as a suspension in an aqueous base or a pharmaceutically acceptable oil base, such as an ester of a long chain fatty acid (e.g., ethyl oleate).

A topical semi-solid ointment formulation typically contains a concentration of the active ingredient from about 1 to 20% (e.g., 5 to 10%) in a carrier such as a pharmaceutical cream base. Various formulations for topical use include drops, tinctures, lotions, creams, solutions, and ointments containing the active ingredient and various supports and vehicles.

The optimal percentage of the inhibitor or antifungal agent in each pharmaceutical formulation varies according to the formulation itself and the therapeutic effect desired in the specific pathologies and correlated therapeutic regimens. Appropriate dosages can readily be determined by those of ordinary skill in the art of medicine by monitoring the patient, animal, or plant for signs of disease amelioration or inhibition, and increasing or decreasing the dosage and/or frequency of treatment as desired. The optimal amount of the compound used for treatment of conditions caused by or contributed to by fungal infections may depend upon the manner of administration, the age and the body weight of the subject, and the condition of the subject to be treated. Generally, the active ingredient is administered at a dosage of 1 to 100 mg/kg of body weight, and typically at a dosage of 1 to 10 mg/kg of body weight.

### EXAMPLES

The invention is further described by the following example, which is not intended to limit the scope of the invention.

### Example 1

### Identification of the OLE1, YOL101c, and YGL039w Promoters as Targets

The promoters of the OLE1, YOL101c, and YGL039w genes each can be used as “target” sequences for determining whether a test compound is an antifungal agent. Conventional RNA profiling studies revealed that yeast cells treated with a known inhibitor of the Fas2 protein, cerulenin, display an increase in the level of mRNA transcripts of the OLE1, YOL101c, and YGL039w genes. Because the activity of these promoters is upregulated by antifungal agents, these promoters are suitable targets, and compounds that increase their activity are expected to be inhibitors of fungal fatty acid biosynthesis.

The OLE1, YOL101c, and YGL039w promoters (along with their respective ATG translation start sites) each were, individually, flised to a lacZ reporter gene (which lacked an ATG translation start site) and introduced in the vector pRS415. The resulting reporter plasmids were then, separately, introduced into a multi-drug resistant yeast strain, thereby producing PL1, P41, and Ml, which are identical to each other except that PL1 carries the YOL101c promoter, P41 carries the OLE1 promoter, and M1 carries the YGL039w promoter (Millennium Pharmaceuticals, Inc., MMB#1495, 1497 and 1604, respectively). These yeast strains can be readily used to measure the level of activity of the OLE1, YOL101c, or YGL039w promoter by measuring expression of the lacZ gene. Enzymatic and colorimetric assays for measuring lacZ gene expression are well known in the art and can be used in the invention.

Treatment of these yeast strains with the Fas2 protein inhibitor cerulenin resulted in an increase in lacZ gene expression, as desired, confirming that inhibitors of Fas2p will increase the activity of the OLE1, YOL101c, and YGL039w promoters. With the OLE1 and YOL101c strains, the induction of promoter activity was detectable with a concentration of cerulenin that was several fold lower than the minimal inhibitory concentration (MIC). A more detailed review of this example and the results are described below.

Transcriptional profiling (TxP) was employed to identify potential candidate genes that would capture fatty acid synthase (FAS) inhibitors in a reporter-based screen. This powerful tool allows for the expression levels of the entire yeast genome to be monitored simultaneously following treatment of yeast cells with a known FAS inhibitor. Logarithmically growing hypersensitive yeast MMB 1489 (Δpdr5 Δsnq2) cells were treated with cerulenin at 0.5 X, 1 X, and 1.5 X the MIC value (0.1, 0.2 and 0.3 μM) and cells were harvested at 1, 3 and 5 hour time points following drug treatment. Total RNA was obtained from cell pellets treated with 0.1, 0.2, and 0.3 μM cerulenin. These RNA samples were profiled along with untreated control samples. cDNA labeling and hybridization against an array containing the yeast genome was completed as described (Chiang, L. et al. P.N.A.S., 98:2814-2819, 2001). Following hybridization, the data was normalized, filtered and analyzed. At the 1 hour time point there were relatively few alterations in gene expression levels, however at 3 and 5 hour time points a clear pattern of gene induction emerged. Among the cerulenin-induced genes at these time points were a number of genes known to be involved in fatty acid biosynthesis, a number of unknown open reading frames (ORFs) plus a number of genes that respond to cellular stress. Three of the most highly induced genes were OLE1 (a Δ-9 fatty acid desaturase, required for synthesis of unsaturated fatty acids), YOL101c, and YGL039w.

The transcriptional profiling results seen in a FAS2 shut-off strain (MMB1258) verified the data observed in cells treated with cerulenin. The FAS2 shut-off strain contains the FAS2 gene under the control of the regulatable MET25 promoter, such that in the presence of 1 mM methionine, expression of the FAS2 gene is repressed. The kinetics of this repression were monitored by viability studies and Northern hybridization (data not shown). According to these studies, it takes 6 to 8 hours to observe growth inhibition after addition of 1 mM methionine. This effectively results in a reduction in the cellular levels of the fatty acid synthetase complex, which converts acetyl- and malonyl-CoA to long chain fatty acids. In addition, the TxP profiles of the shut-off after 8 hours of the addition of 1 mM methionine, similar to those with cerulenin treatment (3-5 hours at IX MIC), showed strong induction with OLE1, YOL101c, and YGL309w (data not shown).

These results confirmed the results of the cerulenin study, that the three genes, OLE1, YOL101c, and YGL039w, are useful reporters for screening compounds for their ability to inhibit fatty acid, etc. synthesis.

### Example 2

### Assays for Candidate Compounds and Antifungal Agents

Having determined that antiflingal agents like cerulenin increase the activity of the OLE1 and YOL101 c promoters, yeast strains containing one or both promoters each operably linked to a reporter gene (e.g., lacZ) can be used in a method for determining whether a test compound is an inhibitor of fungal fatty acid synthesis and is thus a candidate antifungal agent. Such a method can be carried out as follows. Although the following example describes use of the OLE1 promoter, the YOL101c and YGL039w promoters can be used in a similar manner. Persons of ordinary skill in the art recognize that many comparable reagents, apparatuses, and methods can readily be substituted for those described herein.

Yeast Glycerol Stocks: A yeast strain containing the OLE1 promoter operably linked to a reporter gene is grown to an OD₆₀₀ of approximately 0.6. A 60 ml culture of the strain then is centrifuged in a Beckman tabletop centrifuge at 3750 rpm for 10 minutes to pellet the yeast cells. The supernatant then is decanted, and the cell pellet is resuspended in 50 ml of freezing medium (150 ml of SC-Leu medium/50 ml glycerol). The cell suspension then is dispensed into vials in 1 ml aliquots and stored at −80° C.

Screening Plates: Typically, test compounds are tested at two (or more) concentrations for their ability to increase OLE1 promoter activity. For example, the test compounds can be tested at concentrations of 25 μM and 100 μM. In a convenient high throughput screening method, the test compounds can be assayed in the wells of a multiple-well (e.g., 96-well) plastic plate (e.g., Packard, Inc. Cat. # 6005181), with each well containing a different test compound. Thus, one plate can contain the test compounds at a concentration of 25 μM, while another plate contains the test compounds at a concentration of 100 μM. Each well also contains up to 2.5% DMSO.

Cerulenin is used as a positive control in wells lacking a test compound. A stock solution of cerulenin (2,3-Epoxy-4-oxo-7,1-dodecadienamide) can be prepared to a concentration of 10 mg/ml in DMSO and stored at −20° C. When used at a concentration of 2.0 μg/ml, cerulenin provides a control for the maximum induction of promoter activity and maximum inhibition of fungal growth. When used at a concentration of 0.2 μg/ml, cerulenin provides a control for partial induction of promoter activity and no detectable inhibition of fungal growth. A well of the plate that lacks cerulenin (and lacks a test compound) can be used as a negative control, indicating no increase in OLE1 promoter activity and no inhibition of fungal growth.

Induction of OLE1 Promoter Activity: The yeast glycerol stocks described above are used to prepare fresh yeast cultures that are used in the screening assays. The cell suspension, if frozen, is thawed at 37° C., and 33 μl of the stock is added to 12 ml of SC-Leu medium. The culture then is incubated in a shaking Erlemneyer flask at 30° C. overnight (approximately 14-15 hours). The OD₆₀₀ of the culture then is measured, with a culture having an OD₆₀₀ of 0.05-0.15 being suitable for use. Typically, the culture is Attorney's Docket No. 06286-119001 Client's Ref. No. MP12000-157P1R grown to an OD₆₀₀ of 0. 1; if necessary, the culture can be diluted with SC-Leu medium to obtain the desired OD₆₀₀. A 100 μI aliquot of the yeast culture then is added to each well of the 96-well plate containing a test compound, and the cultures are incubated at 30° C. for approximately 6 hours.

To assay for an increase in the activity of the OLE1 promoter, an increase in the level of the lacZ reporter gene expression is measured. A 100 μl aliquot of 2 X substrate buffer is added to each well of the plate. To produce a 100 ml sample of 2 X substrate buffer, the following components are mixed within 1 hour of use, and kept at room temperature: 4.0 ml of GALACTON-STAR™ substrate ((3-chloro-5-(4-methoxyspiro { 1,2-dioxetane-3,2′-(4′chloro)-tricyclo-[3.3.1.1 ^{3,7}] decan} -4-yl)phenyl-B-D-galactopyranoside); Tropix, Inc., Cat. #GS100), 20.0 ml of SAPHIRE II™ luminescence signal enhancer (Tropix, Inc., Cat. #LAX250), and 76 ml of Lysis Buffer (0.026% Na Deoxycholic acid, 0.053% CTAB, 265 mM NaCl, 395 mM HEPES, pH 7.5). The plates are incubated at room temperature for 45 minutes, before vortexing them for 30 seconds at a low to moderate speed.

A chemiluminescent signal is produced by reaction of β-galactosidase with the substrate. Chemiluminescence can be measured using a TopCount-HTS, with the data mode set to measure seconds per count, and with a count time of 0.1 minute. Generally, chemiluminescence should be measured before significant signal decay occurs (i.e., within approximately 90 minutes of initiating the reaction). When cerulenin is used at a concentration of 2.0 μg/ml, providing maximum induction of promoter activity, a 100-fold increase in the level of chemiluminescence is detected, relative to the level obtained with the uninduced control. A chemiluminescent signal that is 2-fold higher than the signal obtained with the uninduced control is sufficient to indicate that the test compound increases the activity of the OLE1 promoter and is an inhibitor of fatty acid synthesis and is thus a candidate antifungal compound.

Assay of Fungal Growth Inhibition: If desired, the ability of each candidate compound to inhibit fungal growth can readily be measured as follows. After incubating the yeast cultures and test compounds for 6 hours in the 96-well plates, as described above, the OD₆₀₀ of the plates is measured (e.g., using a Wallac/Victor 2 plate reader). The OD₆₀₀ can be measured before or after the above-described chemiluminescent assay. A test compound that decreases the OD₆₀₀ of a yeast culture, relative to the OD₆₀₀ obtained in the absence of the candidate compound, is an antifungal agent.

Assay of Inhibition of Fatty Acid, Ergosterol, Sphingolipid, and Phospholipid Synthesis: If desired, candidate compounds that increase promoter activity in the above-described assays can be further tested for their ability to inhibit fatty acid, ergosterol, sphingolipid, or phospholipid synthesis. For example, candidate compounds identified in the above-described assay can be further tested for their ability to inhibit the incorporation of acetate into fatty acids or ergosterol (see, e.g., Buttke et al., Biochemistry, 17:5282-5286, 1978; Kuhajda et al., P.N.A.S., 91:6379-6383, 1994; Weiss et al., Biol. Chem. Hoppe Seyler, 367:905-912, 1986; Stoops et al., J. Biol. Chem., 253:4464-4475, 1978; Duronio et al., J. Cell Biol., 113:1313-1330, 1991; Meyer et al., 117:345-350, 1974; Zhao et al., Microbiol., 142:2509-2514, 1996; and Schneiter et al., Mol. Cell. Biol., 16:7161-7172, 1996, which are incorporated herein by reference).

Generally, ¹⁴C-acetate incorporation is measured essentially as described in Kuhajda et al., P.N.A.S., 91:6379-6383, 1994. Logarithmically growing cells (4⁸ cells of S. cerevisiae MMB1493 or C. albicans SC5314 or Y01.06) were preincubated with 100 μM compounds in DMSO for 10 minutes at 30° C. ¹⁴C-Acetate was added to 20 μCi, mixed and further incubated for 10 minutes at 30° C. Fatty acids were recovered by alkaline ethanolysis (7.5% KOH in 90% ethanol, 1 00ul) at 50° C. for 1 hour, followed by acidification with concentrated HCl (15 μl) and petroleum ether extraction. The radioactivity in the organic layer was counted using a Beckman LS650 multipurpose scintillation counter using Betamax (ICN) scintillation fluid. Cerulenin was used as control at 100 μM; at this concentration 80-90% inhibition was routinely observed in this assay.

In another inhibition assay, Saccharomyces cerevisiae FAS complex was obtained from a yeast extract (Sigma) and assayed as described in (Stoops et al., J.B.C., 253:4464-4475, 1978). A crude preparation of Candida albicans FAS can be prepared from a 1-21 YPD overnight culture of SC5314 following essentially the protocol used for S. cerevisiae FAS, with the exception that the last purification step by gel filtration was omitted. The assay cocktail contains: 0.14 mM NADPH, 0.36 mM acetyl-CoA, 0.18 mM malonyl-CoA, 5 mM EDTA, 8 mM DTT, 0.1 M MES pH 6, 100 μM compound in 1% DMSO, and fungal FAS extract. Changes in NADPH oxidation are followed for 1 hour using a BMG fluorimeter set at 340 nm excitation and 460 nm emission. The inhibition of fatty acid synthesis in these assays is expressed as a percent activity compared to the one obtained using 1% DMSO. Cerulenin as a positive control at 100 μM results in an 80-90% inhibition within the first 30 minutes.

These compounds can also be tested for inhibition of sphingolipid or phospholipid synthesis in an ortho-[³³P]-phosphate assay (see, e.g., Steiner et al., Biochim. et Biophys. Acta, 260:82-87). Any of these biochemical screens can be used to confirm that the identified compounds inhibit fatty acid synthesis.

### Example 3

### Testing of Compounds in High-Throughput Screening Assays

Screening plates containing 80,000 individual small molecule compounds (comprising a diversity file) were prepared at concentrations of 100 μM in 96-well plastic clear-bottom plates (Packard, Inc. Cat. # 6005181). The DMSO concentration was 0.5% (w/v). Cerulenin was used as a positive control at two concentrations. At 9 μM, cerulenin provides a control for the maximum induction of promoter activity and maximum inhibition of fungal growth, and at 0.9 μM cerulenin provides a 50% induction of promoter activity and no detectable inhibition of fungal growth. Empty wells were provided as a negative control, indicating no increase in OLE1 promoter activity and no inhibition of fungal growth.

For the assay, the reporter strains were grown at 30° C. overnight (approximately 14-15 hours) in SC-Leu medium. Typically, the cultures were grown to an OD₆₀₀ of 0.1, though cells at OD₆₀₀ of 0.05-0.15 were also appropriate. Plates were then seeded with a 100 μl aliquot of the yeast culture and incubated at 30° C. for approximately 6 hours. To identify antifungal compounds (those affording growth inhibition), the OD₆₀₀ was measured using a Wallac/Victor 2 plate reader.

To measure the activity of the OLE1 promoter, the amount of β-galactosidase activity in each of the wells was assayed. To that end, a 100 μl aliquot of 2 X β- galactosidase Galacton-Star™ substrate in lysis buffer was added to each well of the plate. The plates were vortexed for 30 seconds at a low to moderate speed and incubated at room temperature for 45-60 minutes. Following this incubation period, chemiluminescence was measured using a TopCount-HTS® (Packard). Signal stability was observed within approximately 90 minutes of initiating the reaction. Under these conditions, cerulenin at 9 μM produced a 100-300 fold increase in chemiluminescence with both promoter strains, however, with the YOL1010 strain the basal level of expression/chemiluminescence was 100-fold lower than that of OLE1. Since some compounds in the library had some chemiluminescent background, the OLE1 sensitivity was superior to that of the YOL101 promoter (data not shown).

The library of 80,000 small molecule compounds was screened for FAS inhibitors at concentration of 100 μM with the Pₒₗₑ₁-lacZ reporter strain. Hits were defined as compounds that afforded 2-fold Pₒₗₑ₁-lacZ induction. The primary screen yielded 850 hits (1.1% hit rate) as defined above. Among these, 182 were deemed structurally acceptable and subjected to retesting using the same Pₒₗₑ₁-IacZ induction assay and an antifungal assay by growth inhibition in MIC testing.

Confirmatory assays identified 65 inducers of which 24 were antifimgal in a S. cerevisiae mdr mutant. Since the OLE1 reporter could capture inhibition in several biosynthetic pathways based on the panel of known antifungal agents, the screen cascade included filtering biochemical assays to select for FAS hits. A FAS assay using partially purified S. cerevisiae FAS complex was used to measure NADPH oxidation as described herein. Among the confirmed antifungal agents, one compound, ML68,216, was identified whose FAS activity strongly suggested inhibition of the fatty acid complex (Table 1). Mechanistically, this inhibitor seems to be specific, because the MIC and IC₅₀ values are comparable.

TABLE 1
Profile of FAS Inhibitor ML-68,216 Compared to Cerulenin

| | OLE1 | | FAS-NADPH | |
|---|---|---|---|---|
| | Fold- | | ox | FAS- |
| | induction | MIC | % Inhibition | NADPH ox |
| Compound | (100 μM) | (μM) | @ 100 μM | IC₅₀ (μM) |
| Cerulenin | 479 | 0.2- | 81 | 0.7-1.3 |
| | | 0.9 | | |
| ML-68,216 | 17 | 100 | 80 | 25-50 |

### OTHER EMBODIMENTS

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether a test compound is an inhibitor of fungal fatty acid, ergosterol, sphingolipid, or phospholipid synthesis, the method comprising:
(i) contacting a yeast cell with a test compound, wherein the yeast cell contains
(a) a promoter, the activity of which is increased in the presence of a compound that inhibits fatty acid synthesis, operably linked to
(b) a reporter gene; and
(ii) measuring activity of the promoter, relative to the level of activity of the promoter in the absence of the test compound, wherein an increase in activity indicates that the test compound is an inhibitor of fungal fatty acid, ergosterol, sphingolipid, or phospholipid synthesis.

2. A method of claim 1, wherein the reporter gene is selected from the group consisting of lacZ, cat, gus, a luciferase gene, and a green fluorescent protein gene.

3. A method of claim 1, wherein measuring activity of the promoter comprises measuring binding of antibodies to a product of the reporter gene.

4. A method of claim 1, wherein measuring activity of the promoter comprises measuring an increase in mRNA transcribed from the reporter gene.

5. A method of claim 1, further comprising determining whether the test compound inhibits fungal fatty acid, ergosterol, sphingolipid, or phospholipid synthesis.

6. A method of claim 5, wherein inhibition of fatty acid, ergosterol, sphingolipid, or phospholipid synthesis is detected as inhibition of incorporation of acetate into fatty acid and ergosterol, or by inhibition of orthophosphate incorporation into sphingolipid and phospholipid.

7. A method of claim 5, wherein inhibition of fungal fatty acid, ergosterol, sphingolipid, or phospholipid synthesis is measured in an extract of the cell.

8. A method for determining whether a test compound is an antifungal agent, the method comprising:
(i) contacting a yeast cell with a test compound, wherein the yeast cell contains
(a) an OLE1, YOL101c, or YGL039w promoter, the activity of which is increased in the presence of a compound that inhibits fatty acid, ergosterol, sphingolipid, or phospholipid synthesis, operably linked to
(b) a reporter gene;
(ii) measuring activity of the promoter, relative to the level of activity of the promoter in the absence of the test compound, wherein an increase in activity indicates that the test compound is a candidate compound that inhibits fungal fatty acid, ergosterol, sphingolipid, or phospholipid synthesis; and
(iii) determining whether the candidate compound inhibits growth of a fungus, wherein an inhibition of fungal growth indicates that the candidate compound is an antifungal agent.

9. A method for determining whether a test compound is an antifungal agent, the method comprising:
(i) contacting a yeast cell with a test compound, wherein the yeast cell contains
(a) a promoter, the activity of which is increased in the presence of a compound that inhibits fatty acid, ergosterol, sphingolipid, or phospholipid synthesis, operably linked to
(b) a reporter gene;
(ii) measuring activity of the promoter, relative to the level of activity of the promoter in the absence of the test compound, wherein an increase in activity indicates that the test compound is a candidate compound that inhibits fungal fatty acid, ergosterol, sphingolipid, or phospholipid synthesis; and
(iii) determining whether the candidate compound inhibits growth of a fungus, wherein an inhibition of fungal growth indicates that the test compound is an antifungal agent.

10. A method for determining whether a test compound is a candidate antifungal compound, the method comprising:
(i) contacting a yeast cell with a test compound, wherein the yeast cell contains
(a) an OLE1, YOL101c, or YGL039w promoter, the activity of which is increased in the presence of a compound that inhibits fatty acid, ergosterol, sphingolipid, or phospholipid synthesis, operably linked to
(b) a reporter gene; and
(ii) measuring activity of the promoter, relative to the level of activity of the promoter in the absence of the test compound, wherein an increase in activity indicates that the test compound is a candidate antifungal compound.

11. A method for treating a fungal infection in an organism, the method comprising administering an effective amount of an antifungal agent identified by the method of claim 9 to an organism having a fungal infection, thereby treating the fungal infection in the organism.

12. A method of claim 11, wherein the organism is a mammal.

13. A method of claim 12, wherein the mammal is a human.

14. A method of claim 11, wherein the fungal infection is caused by a fungus selected from the group consisting Candida, Cryptococcus, Aspergillus, or Saccharomyces.

15. A method of claim 11, wherein the fungal infection is an infection caused by a pathogenic fungus.

16. A method of claim 11, wherein the fungal infection is an infection caused by a non-pathogenic fungus.

17. A pharmaceutical formulation comprising an antifungal agent identified by the method of claim 9 and a pharmaceutically acceptable carrier.

18. A method of preparing an inhibitor of fungal fatty acid, ergosterol, sphingolipid, or phospholipid synthesis, the method comprising:
screening multiple test compounds by the method of claim 1;
identifying candidate compounds that upregulate promoter activity;
isolating one or more lead compounds from the candidate compounds;
identifying and selecting a lead compound that inhibits fungal fatty acid, ergosterol, sphingolipid, or phospholipid synthesis; and
formulating the selected lead compound as an inhibitor of fungal fatty acid, ergosterol, sphingolipid, or phospholipid synthesis.

19. A method of preparing an antifungal agent, the method comprising:
screening multiple test compounds by the method of claim 9;
identifying candidate compounds that upregulate promoter activity;
isolating one or more lead compounds from the candidate compounds;
identifying and selecting a lead compound that inhibits growth of a fungus; and
formulating the selected lead compound as an antifungal agent.

20. A method for preparing an inhibitor of fungal fatty acid, ergosterol, sphingolipid, or phospholipid synthesis, the method comprising:
screening multiple test compounds by the method of claim 1;
identifying candidate compounds that upregulate promoter activity;
isolating one or more lead compounds from the candidate compounds;
derivatizing said one or more lead compounds, thereby producing derivatives;
identifying derivatives that inhibit fungal fatty acid synthesis; and
formulating the derivative as an inhibitor of fungal fatty acid, ergosterol, sphingolipid, or phospholipid synthesis.

21. An inhibitor of fungal fatty acid, ergosterol, sphingolipid and phospholipid synthesis prepared by the method of claim 20.

22. A method for inhibiting fungal fatty acid, ergosterol, sphingolipid, or phospholipid synthesis in an organism having a fungal infection, the method comprising administering an effective amount of an inhibitor of claim 21 to an organism having a fungal infection, thereby inhibiting fungal fatty acid synthesis in the organism.

23. A method for preparing an antifungal agent, the method comprising:
screening multiple test compounds by the method of claim 9;
identifying candidate compounds that upregulate promoter activity;
isolating one or more lead compounds from the candidate compounds;
derivatizing said one or more lead compounds, thereby producing derivatives;
identifying derivatives that inhibit growth of a fungus; and
formulating the derivative as an antifungal agent.

24. An antifungal agent prepared by the method of claim 23.

25. A pharmaceutical formulation comprising an antifungal agent of claim 24 and a pharmaceutically acceptable carrier.

26. A method for inhibiting growth of fungi in an organism having a fungal infection, the method comprising administering an effective amount of a pharmaceutical formulation of claim 25 to an organism having a fungal infection, thereby inhibiting growth of fungi in the organism.

27. A method of claim 1, wherein the promoter is an OLE1, YOL101c, or YGL039w promoter.
